(19) United States
(12) Patent Application Publication
(10) Pub. No.: US 2023/0210807 A1
(43) Pub. Date: Jul. 6, 2023

(54) USE OF BENZOATE COMPOUND IN TREATMENT OF SARS-COV-2 INFECTIONS

(71) Applicant: ACADEMY OF MILITARY MEDICAL SCIENCES, Beijing (CN)

(72) Inventors: Wu ZHONG, Beijing (CN); Ruiyuan CAO, Beijing (CN); Gengfu XIAO, Beijing (CN); Zhihong HU, Beijing (CN); Manli WANG, Beijing (CN); Leike ZHANG, Beijing (CN); Wei LI, Beijing (CN); Yuexiang LI, Beijing (CN); Lei ZHAO, Beijing (CN); Shiyong FAN, Beijing (CN); Song LI, Beijing (CN)

(21) Appl. No.: 17/793,959

(22) PCT Filed: Jul. 15, 2020
(86) PCT No.: PCT/CN2020/102070

§ 371 Date: Jul. 20, 2022

(30) Foreign Application Priority Data
Jan. 21, 2020 (CN) 202010073050.8

(51) Int. Cl. A61K 31/245; A61P 31/14

(52) U.S. Cl. CPC A61K 31/245; A61P 31/14

(57) ABSTRACT

Disclosed are the use of a benzoate compound as shown in formula I, a geometric isomer thereof, a pharmaceutically acceptable salt thereof and/or a solvate thereof or a hydrate thereof, and a pharmaceutical composition containing the above-mentioned compound in the prevention and treatment of SARS-CoV-2 infections.

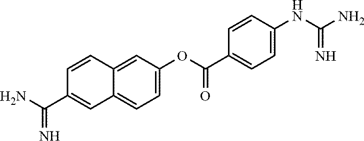 [SMILES: N=C(N)Nc1ccc(C(=O)Oc2ccc3cc(C(=N)N)ccc3c2)cc1]

The invention is based on and claims the benefit of priority from Chinese application No. 202010073050.8, filed on Jan. 21, 2020, the disclosure of which are incorporated herein by reference in its entirety.

### TECHNICAL FIELD

The present application relates to use of a compound represented by Formula I, a geometric isomer, a pharmaceutically acceptable salt and/or a solvate and/or a hydrate thereof, and a pharmaceutical composition comprising the above-mentioned compound in the treatment of a SARS-CoV-2 infection.

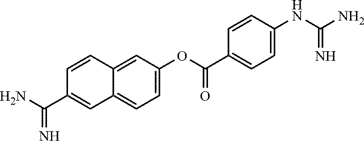 [SMILES: N=C(N)Nc1ccc(C(=O)Oc2ccc3cc(C(=N)N)ccc3c2)cc1]

### BACKGROUND ART

The benzoate compound represented by Formula I, also known as Nafamostat, has a chemical name of 6-amidino-2-naphthyl-4-guanidino benzoate, was originally developed by Torii Pharma of Japan in 1986, its indications are: acute pancreatitis, disseminated intravascular coagulation (DIC), anticoagulant.

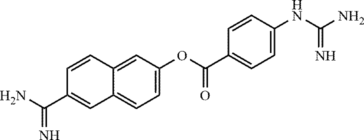 [SMILES: N=C(N)Nc1ccc(C(=O)Oc2ccc3cc(C(=N)N)ccc3c2)cc1]

Nafamostat is a fast-acting proteolytic inhibitor, which prevents the proteolysis of fibrinogen into fibrin during hemodialysis by competitively inhibiting multiple serine proteases including thrombin. It improves acute pancreatitis, prevents blood clot formation in extracorporeal circulation and has anti-inflammatory effects in vitro. Nafamostat has neuroprotective effects in cerebral ischemic injury induced by antithrombin activity. In addition, Nafamostat has also been reported to have multiple pharmacodynamic activities: antiproliferative effects, inhibition of cell adhesion and invasion, and increased anoikis sensitivity were observed in Nafamostat-treated cancer cells; Nafamostat can effectively inhibit the up-regulation of PD-L1 at the messenger RNA (mRNA) and protein levels in human lung cancer cells (HLC-1) induced by interferon-y ray (IFN-γ); Nafamostat exerts therapeutic effects in allergic airway inflammation not only because of its inhibitory effects in the early stages of mast cell activation, but also because of its immunomodulatory functions in the later stages of allergic inflammation. In terms of antivirus, Nafamostat is identified as a membrane fusion inhibitor of MERS coronavirus S protein, which is superior to several other clinically approved serine protease inhibitors. Nafamostat can also block MERS-CoV infection in vitro, and in vitro experiments show that its IC₅₀ is 0.1 µM, which is 10 times higher than that of Camostat.

The 2019 novel coronavirus (2019-nCoV) is a new coronavirus strain that has never been found in humans before. On Feb. 11, 2020, the International Committee on Taxonomy Viruses (ICTV) announced that the official name of 2019 novel Coronavirus (2019-nCoV) is called severe acute respiratory syndrome coronavirus 2 (SARS-CoV-2). On the same day, the World Health Organization (WHO) announced that the official name of the disease caused by this virus is COVID-19. The symptoms of SARS-CoV-2 virus infection are mainly pneumonia, and can be divided into simple infection, mild pneumonia, severe pneumonia, acute respiratory distress syndrome, sepsis, septic shock and so on according to the severity of disease. Patients with simple infection may have non-specific symptoms, such as fever, cough, sore throat, nasal congestion, fatigue, headache, muscle pain or discomfort, and the elderly and immunosuppressed people may have atypical symptoms. Patients with mild pneumonia mainly have cough, dyspnea and polypnea. Severe pneumonia can be seen in adolescents, adults or children, and the main symptoms of which include increased breathing frequency, severe respiratory failure or dyspnea, central cyanosis, drowsiness, unconsciousness or convulsion, gasp, etc. The lung images of acute respiratory distress syndrome are bilateral ground glass shadows, which cannot be completely explained by effusion, lobar exudation or atelectasis or lung mass shadows, and the main symptom of which is pulmonary edema. Patients with sepsis often have fatal organ dysfunction, and the most critical patients are those with septic shock, and they may have a high probability of death. At present, the SARS-CoV-2 infection is mainly treated with supportive therapy in clinic, and no specific antiviral drug is available.

### CONTENTS OF THE INVENTION

The purpose of the present application is to discover a compound with an antiviral activity against SARS-CoV-2, so as to develop a drug for the treatment of SARS-CoV-2 infection. Through creative research in the present application, it is found that the compound represented by Formula I has a function of inhibiting the replication of SARS-CoV-2, and has a good potential therapeutic effect in the treatment of a disease caused by a SARS-CoV-2.

The present application provides a compound represented by Formula I, a geometric isomer, a pharmaceutically acceptable salt and/or a solvate or a hydrate thereof:

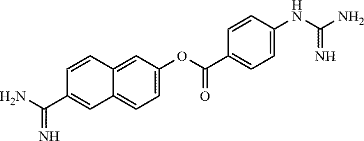 [SMILES: N=C(N)Nc1ccc(C(=O)Oc2ccc3cc(C(=N)N)ccc3c2)cc1]

According to the present application, the pharmaceutically acceptable salts of the compound represented by Formula I of the present application include an inorganic or organic acid salt thereof and an inorganic or organic base salt thereof. The present application relates to all forms of the above salts, including but not limited to: sodium salt, potassium salt, calcium salt, lithium salt, meglumine salt, hydrochloride salt, hydrobromide salt, hydroiodide salt, nitrate salt, sulfate, hydrogen sulfate, phosphate, hydrogen phosphate, acetate, propionate, butyrate, oxalate, pivalate, adipate, alginate, lactate, citrate, tartrate, succinate, maleate, fumarate, picrate, aspartate, gluconate, benzoate, methanesulfonate, ethanesulfonate, benzenesulfonate, p-toluenesulfonate and embonate and so on.

In some embodiments, the pharmaceutically acceptable salt of the compound represented by Formula I is a methanesulfonate of Nafamostat.

According to the present application, the compound represented by Formula I can inhibit the replication of SARS-CoV-2 in a cell and reduce the nucleic acid load of SARS-CoV-2 in a cell culture.

After creative invention and research, the inventors of the present application have discovered some new features of the compound represented by Formula I: the compound represented by Formula I can reduce the viral nucleic acid load in SARS-CoV-2 infected cells at micromolar concentration level.

The present application also relates to use of the compound represented by Formula I, a geometric isomer, a pharmaceutically acceptable salt and/or a solvate and/or a hydrate thereof in the manufacture of a medicament for the prevention and/or the treatment of a disease or an infection caused by a SARS-CoV-2 (including but not limited to a respiratory disease (e.g., simple infection (such as fever, cough and sore throat), pneumonia, acute or severe acute respiratory infection, hypoxic respiratory failure and acute respiratory distress syndrome), sepsis and septic shock, etc.),

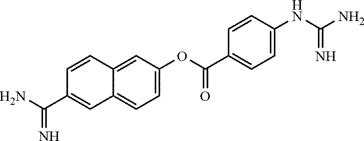 [SMILES: N=C(N)Nc1ccc(C(=O)Oc2ccc3cc(C(=N)N)ccc3c2)cc1]

The present application also relates to use of the compound represented by Formula I, a geometric isomer, a pharmaceutically acceptable salt and/or a solvate and/or a hydrate thereof in the manufacture of a medicament as a SARS-CoV-2 inhibitor.

The present application also relates to use of the compound represented by Formula I, a geometric isomer, a pharmaceutically acceptable salt and/or a solvate and/or a hydrate thereof in the manufacture of a medicament for inhibiting the replication or reproduction of SARS-CoV-2 in a cell (e.g., a cell of mammal).

The present application also relates to a pharmaceutical composition, which comprises the compound represented by Formula I, a geometric isomer, a pharmaceutically acceptable salt and/or a solvate and/or a hydrate thereof, preferably, the pharmaceutical composition further comprises a pharmaceutically acceptable carrier or excipient; specifically, the pharmaceutical composition is a solid preparation, an injection, an external preparation, a spray, a liquid preparation, or a compound preparation.

The present application also relates to use of the pharmaceutical composition comprising the compound represented by Formula I, a geometric isomer, a pharmaceutically acceptable salt and/or a solvate and/or a hydrate thereof or the compound represented by Formula I, a geometric isomer, a pharmaceutically acceptable salt and/or a solvate and/or a hydrate thereof in the manufacture of a medicament for the prevention and/or the treatment of a disease including but not limited to a respiratory disease (simple infection (such as fever, cough and sore throat), pneumonia, acute or severe acute respiratory infection, hypoxic respiratory failure and acute respiratory distress syndrome), sepsis and septic shock, etc.

The present application also relates to use of the pharmaceutical composition in the manufacture of a medicament for the prevention and/or the treatment of a disease or an infection caused by a SARS-CoV-2 (e.g., a respiratory disease (e.g., simple infection (such as fever, cough and sore throat), pneumonia, acute respiratory infection or severe acute respiratory infection, hypoxic respiratory failure and acute respiratory distress syndrome), sepsis and septic shock, etc.

The present application also relates to use of the pharmaceutical composition in the manufacture of a medicament as a SARS-CoV-2 inhibitor.

The present application also relates to use of the pharmaceutical composition in the manufacture of a medicament for inhibiting the replication or reproduction of SARS-CoV-2 in a cell (e.g., a cell of mammal).

The present application also relates to a method for preventing and/or treating a disease in a mammal in need thereof or a method for inhibiting the replication or reproduction of SARS-CoV-2 in a mammal in need thereof, wherein the method comprises administering to the mammal in need thereof a therapeutically and/or prophylactically effective amount of a pharmaceutical composition comprising a compound represented by Formula I, a geometric isomer, a pharmaceutically acceptable salt and/or a solvate and/or a hydrate thereof, or a compound represented by Formula I, a geometric isomer, a pharmaceutically acceptable salt and/or a solvate and/or a hydrates thereof, wherein the disease includes a disease or an infection caused by a SARS-CoV-2 (e.g., severe acute respiratory infection (SARI)).

In some embodiments, the disease or the infection caused by a SARS-CoV-2 of the present application includes but not is limited to a respiratory disease (e.g., simple infection (such as fever, cough and sore throat), pneumonia, acute respiratory infection or severe acute respiratory infection, hypoxic respiratory failure and acute respiratory distress syndrome), sepsis and septic shock, etc.

The present application also relates to the compound represented by Formula I, a geometric isomer, a pharmaceutically acceptable salt and/or a solvate and/or a hydrate thereof, for use in preventing and/or treating a disease or an infection caused by a SARS-CoV-2 (including but not limited to a respiratory disease (e.g., simple infection (such as fever, cough and sore throat), pneumonia, acute respiratory infection or severe acute respiratory infection, hypoxic respiratory failure and acute respiratory distress syndrome), sepsis and septic shock, etc.).

The present application also relates to the compound represented by Formula I, a geometric isomer, a pharmaceutically acceptable salt and/or a solvate and/or a hydrate thereof, for use as a SARS-CoV-2 inhibitor.

The present application also relates to the compound represented by Formula I, a geometric isomer, a pharmaceutically acceptable salt and/or a solvate and/or a hydrate thereof, for use in inhibiting the replication or reproduction of SARS-CoV-2 in a cell (e.g., a cell of mammal).

The present application also relates to the pharmaceutical composition, for use in preventing and/or treating a disease or an infection caused by a SARS-CoV-2 (e.g., a respiratory disease (e.g., simple infection (such as fever, cough and sore throat), pneumonia, acute respiratory infection or severe acute respiratory infection, hypoxic respiratory failure and acute respiratory distress syndrome), sepsis and septic shock, etc.).

The present application also relates to the pharmaceutical composition, for use as a SARS-CoV-2 inhibitor.

The present application also relates to the pharmaceutical composition, for use in inhibiting the replication or reproduction of SARS-CoV-2 in a cell (e.g., a cell of mammal).

In some embodiments, the disease caused by a SARS-CoV-2 described in the present application is COVID-19.

In the present application, the official name of the term “2019 novel coronavirus (2019-nCoV)” is severe acute respiratory syndrome coronavirus 2 (SARS-CoV-2).

In the present application, the official name of the term “disease caused by 2019 novel coronavirus (2019-nCoV)” is COVID-19.

In some embodiments, the mammal includes bovine, equine, caprid, suidae, canine, feline, rodent, primate, wherein the preferred mammal is a human, a cat, a dog, or a pig.

The pharmaceutical composition described in the present application can be prepared into various forms according to different administration routes.

According to the present application, the pharmaceutical composition can be administered in any one of the following routes: oral administration, spray inhalation, rectal administration, nasal administration, buccal administration, vaginal administration, topical administration, parenteral administration such as subcutaneous, intravenous, intramuscular, intraperitoneal, intrathecal, intraventricular, intrasternal and intracranial injection or infusion, or administration with the help of an explant reservoir. Among them, oral, intraperitoneal or intravenous administration is preferred.

When orally administered, the compound represented by Formular I, a geometric isomer, a pharmaceutically acceptable salt and/or a solvate and/or a hydrate thereof can be prepared into any form of orally acceptable preparation, including but not limited to a tablet, a capsule, an aqueous solution or an aqueous suspension. Generally, the carrier for use in a tablet includes lactose and corn starch, and a lubricant such as magnesium stearate can also be added. The diluent for use in a capsule generally includes lactose and dry corn starch. The aqueous suspension is usually used by mixing an active ingredient with a suitable emulsifier and a suitable suspending agent. If necessary, a sweetener, a flavoring agent or a coloring agent can also be added to the above-mentioned forms of oral preparation.

When rectally administered, the compound represented by Formular I, a geometric isomer, a pharmaceutically acceptable salt and/or a solvate, and/or a hydrate thereof can generally be prepared in a form of suppository, which is prepared by mixing the drug with a suitable non-irritating excipient. The excipient is present in solid state at room temperature, but melts at the rectal temperature to release the drug. Such excipient includes cocoa butter, beeswax and polyethylene glycol.

When topically administered, especially for the treatment of easily accessible affected-surface or organ, such as eye, skin, or lower intestinal neurological disease by topical application, the compound represented by Formular I, a geometric isomer, a pharmaceutically acceptable salt and/or a solvate and/or a hydrate thereof can be prepared in various forms of topical preparations according to different affected-surfaces or organs, the specific instructions are as follows:

When topically administered to eye, the compound represented by Formular I, a geometric isomer, a pharmaceutically acceptable salt and/or a solvate and/or a hydrate thereof can be formulated into a preparation form such as micronized suspension or solution, the carrier used is isotonic sterile saline with a certain pH, and a preservative such as benzyl chloride alkoxide may or may not be added. In addition, for administration to eye, the compound can also be prepared in a form of ointment such as vaseline ointment.

When topically administered to skin, the compound represented by Formular I, a geometric isomer, a pharmaceutically acceptable salts and/or a solvate and/or a hydrate thereof can be prepared into a suitable form such as an ointment, a lotion or a cream, in which the active ingredient is suspended or dissolved in one or more carriers. The carrier for use in an ointment includes, but is not limited to mineral oil, liquid petrolatum, white petrolatum, propylene glycol, polyethylene oxide, polypropylene oxide, emulsifying wax, and water. The carrier for use in a lotion or a cream includes, but is not limited to mineral oil, sorbitan monostearate, Tween-60, cetyl ester wax, hexadecenyl aryl alcohol, 2-octyldodecanol, benzyl alcohol and water.

When topically administered to lower intestinal tract, the compound represented by Formular I, a geometric isomer, a pharmaceutically acceptable salt and/or a solvate and/or a hydrate thereof can be prepared into a form such as rectal suppository as described above or a suitable enema preparation form, in addition, a topical transdermal patch can also be used.

The compound represented by Formular I, a geometric isomer, a pharmaceutically acceptable salt and/or a solvate and/or a hydrate thereof can also be administered in a preparation form of sterile injection, including sterile injectable aqueous solution or oil suspension, or sterile injectable solutions, wherein the usable carrier and solvent includes water, Ringer’s solution and isotonic sodium chloride solution. In addition, a sterilized non-volatile oil such as monoglyceride or diglyceride can also be used as solvent or suspension media.

The drugs of the above various preparation forms can be prepared according to conventional methods in the pharmaceutical field.

In the present application, the term “therapeutically effective amount” or “prophylactically effective amount” refers to an amount that is sufficient to treat or prevent a patient’s disease but is sufficiently low to avoid serious side effects (at a reasonable benefit/risk ratio) within a reasonable medical judgment. The therapeutically or prophylactically effective amount of the compound will change according to the factors such as the selected specific compound (e.g., considering the efficacy, effectiveness, and half-life of compound), the selected administration route, the disease to be treated or prevented, the severity of the disease to be treated or prevented, the treated or prevented patient’s age, size, weight and physical disease, medical history, duration of treatment or prevention, nature of concurrent therapy, desired therapeutic or prophylactic effect, etc., but can still be routinely determined by those skilled in the art.

In addition, it should be noted that the specific dosage and method of using the compound represented by Formular I, a geometric isomer, a pharmaceutically acceptable salt and/or a solvate and/or a hydrate thereof for different patients depends on many factors, including the patient’s age, weight, gender, natural health status, nutritional status, active strength of drug, administration time, metabolic rate, severity of disease, and subjective judgment of physician. Herein it is preferred to use a dosage between 0.0001-1000 mg/kg body weight/day.

### BRIEF DESCRIPTION OF THE DRAWINGS

FIG. 1 shows that Nafamostat can effectively reduce the viral nucleic acid load in Vero E6 cells infected by SARS-CoV-2. In FIG. 1, (a) shows that Nafamostat can reduce the viral RNA load in the cells 48 hours after the cells were infected by SARS-CoV-2. The ordinate is the copy number of viral RNA in the sample, and the abscissa is the drug concentration; (b) shows that the test cells were treated by Nafamostat at the test concentration for 48 hours, and no cytotoxicity was observed. The ordinate is the percentage of cell viability relative to the vehicle control group (only cells, no drug was added), and the abscissa is the drug concentration.

### SPECIFIC MODELS FOR CARRYING OUT THE INVENTION

The following examples are illustrative preferred embodiments of the present application and do not constitute any limitation to the present application.

Example 1: Experiment on Nafamostat represented by Formular I reducing viral nucleic acid load in SARS-CoV-2 infected cells

### Drug Treatment of Virus-Infected Cells

Vero E6 cells (purchased from ATCC, Catalog No. 1586) was inoculated on a 24-well plate, cultured for 24 hours; then virus infection was carried out. Specifically, SARS-CoV-2 (2019-nCoV) virus (nCoV-2019BetaCoV/Wuhan/WIV04/2019 strain, provided by Wuhan Institute of Virology, Chinese Academy of Sciences) was diluted with 2% cell maintenance solution (formulation: FBS (purchased from Gibco company, Catalog No. 16000044) was added to MEM (purchased from Gibco, Catalog No. 10370021) at a volume ratio of 2%, thereby obtaining the 2% cell maintenance solution) to a corresponding concentration, and then added to a 24-well plate so that each well contained a viral load of 100TCID₅₀. Nafamostat (purchased from MedChemExpress, Catalog No. HY-B0190) were diluted with 2% cell maintenance solution to corresponding concentrations and added separately to the corresponding wells, so that the final concentrations of the drugs were 100 µM, 33 µM, 11 µM, 3.7 µM, 1.23 µM, 0.41 µM, 0.14 µM, respectively, then the plate was placed in a 37° C., 5% CO₂ incubator and cultured for 48 hours. To the vehicle control group, the 2% cell maintenance solution without any test drugs was added.

### RNA Extraction

RNA extraction kit was purchased from Qiagen Company, Catalog No. 74106. The consumables (spin columns, RNase-free 2 ml collection tubes, etc.) and reagents (RLT, RW1, RPE, RNase-free water, etc.) involved in the following RNA extraction steps were part of the kit. The following extraction steps were recommended steps in the kit instruction.

1) 100 µL of the supernatant was taken from the tested plate and added to a nuclease-free EP tube, then 350 µL of Buffer RLT was added to each well and mixed by beating with a transfer liquid gun until complete lysis was achieved, then centrifugation was carried out to obtain a supernatant;

2) an equal volume of 70% ethanol was added to the supernatant obtained in 1) and mixed well;

3) the mixture solution obtained in 2) was transferred to a RNase-free spin column, and centrifuged at 12000 rpm for 15 seconds, and the waste liquid was discarded;

4) 700 µL of Buffer RW1 was added to the spin column, and centrifuged at 12000 rpm for 15 seconds to clean the spin column, and the waste liquid was discarded;

5) 500 µL of Buffer RPE was added to the spin column, and centrifuged at 12000 rpm for 15 seconds to clean the spin column, and the waste liquid was discarded;

6) 500 µL of Buffer RPE was added to the spin column, and centrifuged at 12000 rpm for 2 min to clean the spin column, the waste was discarded;

7) a new RNase-free 2 ml collection tube was used for replacement, centrifugation was carried out at 12000 rpm for 1 min, the spin column was dried, and then the spin column was transferred to a 1.5 ml collection tube in step 8);

8) a new 1.5 ml collection tube was used for replacement, in which the spin column dried in step 7) was placed, and 30 µl of RNase-free water was added to the spin column, and centrifugation was carried out at 12000 rpm for 2 minutes, the obtained eluate contained the corresponding RNA, then the RNase inhibitor (purchased from NEB company, Catalog No. M0314L) was added, and Nano Drop (purchased from Thermo scientific, Nano Drop One) was used to detect each RNA concentration.

### RNA Reverse Transcription

In the experiment, the reverse transcription kit (PrimeScript™ RT reagent Kit with gDNA Eraser, Catalog No. RR047Q) produced by TaKaRa was used for RNA reverse transcription. The steps were as follows.

① Removal of gDNA: RNA samples of each experimental group were collected, 1 µg of each sample was taken for reverse transcription. First, 2 µl of 5× gDNA Eraser Buffer was added to the RNA sample of each experimental group, the reaction system was supplemented with RNase-free water to reach 10 µl, mixed well, and subjected to water bath at 42° C. for 2 min to remove the gDNA that might be present in the sample;

② Reverse transcription: Appropriate amounts of enzyme, primer Mix and reaction buffer were added to the sample obtained in ①, RNase-free water was added to supplement to reach a volume of 20 µl, the reaction was performed in a water bath at 37° C. for 15 minutes, and then in water bath at 85° C. for 5 seconds, to obtain cDNA by transcription.

### Real-Time PCR

Fluorescence quantitative PCR was used to detect the number of copies per milliliter of the original virus solution. The reaction system was mixed by using TB Green Premix (Takara, Cat#RR820A), and the amplification reaction and reading were carried out with StepOne Plus Real-time PCR instrument (brand: ABI). The copy number contained in per milliliter of the original virus solution was calculated. The steps were as follows:

① Establishment of standard product: the plasmid pMT-RBD (the plasmid was provided by Wuhan Institute of Virology, Chinese Academy of Sciences) was diluted to 5×10⁸ copies/µL, 5×10⁷ copies/µL, 5×10⁶ copies/µL, 5×10⁵ copies/ µL, 5×10⁴ copies/µL, 5×10³ copies/µL, 5×10² copies/µL, respectively. 2 µL of standard product or cDNA template was taken for qPCR reaction.

② The primer sequences used in the experiment were as follows (all indicated in the 5′-3′ direction):

RBD-qF:CAATGGTTTAACAGGCACAGG

RBD-qR:CTCAAGTGTCTGTGGATCACG

③ The reaction procedure was as follows:
Pre-denaturation: 95° C. for 5 minutes;
Cycle parameters: 95° C. for 15 seconds, 54° C. for 15 seconds, 72° C. for 30 seconds, a total of 40 cycles.

### Cytotoxicity Test of Drugs

The cytotoxicity test of drugs was carried out by using CCK-8 kit (Beyotime). Specific steps were as follows:
① 1×10⁴ Vero-E6 cells (ATCC) were inoculated in a 96-well plate and incubated at 37° C. for 8 hours.
② The drug was diluted with DMSO to an appropriate concentration of mother solution, and then diluted with MEM (purchased from Gibco, Catalog No. 10370021) medium containing 2% FBS (purchased from Gibco company, Catalog No. 16000044) to the same concentration as that for the drug treatment. The original medium in the 96-well plate was discarded, 100 µL of the drug-containing MEM medium was taken and added to the cells, and three replicate wells were set for each concentration. A vehicle control (adding DMSO and medium to cells in wells, without adding drug) and a blank control (adding DMSO and medium to the wells, without cells) were set up. After the drug was added, the cells were incubated at 37° C. for 48 hours.
③ 20 µL of CCK-8 solution (Beyotime) was added to the well to be tested, mixed gently without generating bubbles, and incubated subsequently at 37° C. for 2 hours. OD₄₅₀ was read on a microplate reader (purchased from Molecular Devices, model: SpectraMax M5), and the cell viability was calculated:
$$\begin{matrix}\text{Cell} \text{viability} \left(\text{\%}\right) \text{=} \left({\text{A}}_{\text{(drug} \text{treatment} \text{group)}} {\text{-A}}_{\text{(blank} \text{control)}}\right) / \\ \left({\text{A}}_{\text{(vehicle} \text{control)}} {\text{-A}}_{\text{(blank} \text{control)}}\right) \times 100 \%\end{matrix}$$

Wherein, A was the reading of the microplate reader.

### Experimental Results

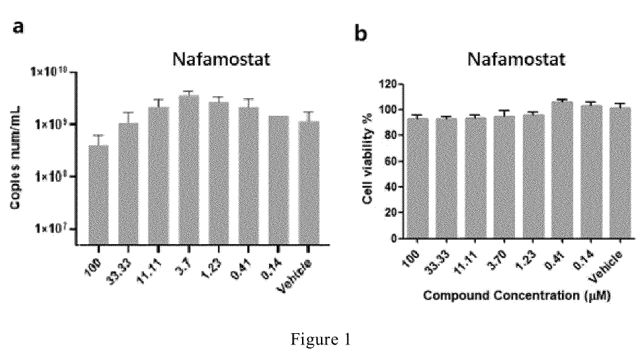

The results of the virus proliferation inhibition experiment showed that the test compound at concentrations of 100 µM, 33 µM, 11.1 µM and 3.7 µM could effectively inhibit the replication of SARS-CoV-2 virus genome in the infected supernatant (Table 1 and FIG. 1).

TABLE 1

Antiviral test results of the test compund (Nafamostat)

| | | | | | | | | |
|---|---|---|---|---|---|---|---|---|
| Concentration (µM) | 100 | 33.33 | 11.11 | 3.70 | 1.23 | 0.41 | 0.14 | Vehicle |
| Copy number of virus genome | 395904281±285024470.56 | 1015568394.5±70116949.22 | 2116737884±785155013.49 | 3601892897.5±680084251.56 | 2805092897.49±419609425.32 | 2051893138.5±1115069812.46 | 1169068894.5±28678551.13 | 1009560294.5±600518414.22 |

The cytotoxicity test results showed that the treatment of the test compound Nafamostat did not change the cell viability at all test concentrations, that was, the test compound had no toxic effect on the cells at all concentrations (Table 2 and FIG. 1).

TABLE 2

Cytotoxicity test results of the test compound (Nafamostat)

| | | | | | | | | |
|---|---|---|---|---|---|---|---|---|
| Concentration (µM) | 100 | 33.33 | 11.11 | 3.70 | 1.23 | 0.41 | 0.14 | Vehicle |
| Cell viability (% of vehicle control) | 92.65±3.37 | 92.64±2.16 | 93.68±1.83 | 94.88±4.44 | 95.85±2.57 | 106.14±1.77 | 103.25±2.53 | 101.17±3.85 |

What is claimed is:

1. (canceled)

2. (canceled)

3. (canceled)

4. (canceled)

5. (canceled)

6. (canceled)

7. A method for preventing and/or treating a disease in a mammal in need thereof, wherein the method comprises administering to the mammal in need thereof a therapeutically and/or prophylactically effective amount of i) a pharmaceutical composition comprising a compound represented by Formula I, a geometric isomer, a pharmaceutically acceptable salt and/or a solvate and/or a hydrate thereof, or ii) a compound represented by Formula I, a geometric isomer, a pharmaceutically acceptable salt and/or a solvate and/or a hydrates thereof, 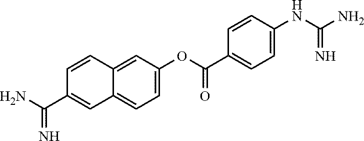
wherein, the disease is a disease or an infection caused by a SARS-CoV-2.

8. The method according to claim 7, wherein the disease or the infection caused by a SARS-CoV-2 is a respiratory disease, sepsis, or septic shock.

9. A method for inhibiting the replication or reproduction of SARS-CoV-2 in a mammal in need thereof, wherein the method comprises administering to the mammal in need thereof a therapeutically and/or prophylactically effective amount of i) a pharmaceutical composition comprising a compound represented by Formula I, a geometric isomer, a pharmaceutically acceptable salt and/or a solvate and/or a hydrate thereof, or ii) a compound represented by Formula I, a geometric isomer, a pharmaceutically acceptable salt and/or a solvate and/or a hydrates thereof,

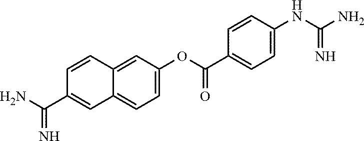 [SMILES: N=C(N)Nc1ccc(C(=O)Oc2ccc3cc(C(=N)N)ccc3c2)cc1]

10. (canceled)

11. (canceled)

12. (canceled)

13. (canceled)

14. (canceled)

15. (canceled)

16. The method according to claim 7, wherein the disease caused by a SARS-CoV-2 is COVID-19.

17. The claim method according to claim 9, wherein the mammal is bovine, equine, caprid, suidae, canine, feline, rodent, or primate .

18. The method according to claim 7, wherein the pharmaceutically acceptable a hydrochloride salt, hydrobromide salt, hydroiodide salt, nitrate salt, sulfate, hydrogen sulfate, phosphate, hydrogen phosphate, acetate, propionate, butyrate, oxalate, pivalate, adipate, alginate, lactate, citrate, tartrate, succinate, maleate, fumarate, picrate, aspartate, gluconate, benzoate, methanesulfonate, ethanesulfonate, benzenesulfonate, p-toluenesulfonate, or embonate of the compound.

19. The method according to claim 18, wherein the pharmaceutically acceptable salt of the compound represented by Formula I is a methanesulfonate of the compound.

20. The method according to claim 7, wherein the disease or the infection caused by a SARS-CoV-2 is severe acute respiratory infection (SARI).

21. The method according to claim 7, wherein the disease or the infection caused by a SARS-CoV-2 is simple infection, fever, cough, sore throat, pneumonia, acute respiratory infection, severe acute respiratory infection, hypoxic respiratory failure or acute respiratory distress syndrome.

22. The method according to claim 9, wherein the mammal is a human, a cat, a dog, or a pig.

23. The method according to claim 7, wherein the pharmaceutical composition further comprises a pharmaceutically acceptable carrier or excipient.

24. The method according to claim 23, wherein the pharmaceutical composition is a solid preparation, an injection, an external preparation, a spray, a liquid preparation, or a compound preparation.

25. The method according to claim 9, wherein the pharmaceutical composition further comprises a pharmaceutically acceptable carrier or excipient.

26. The method according to claim 25, wherein the pharmaceutical composition is a solid preparation, an injection, an external preparation, a spray, a liquid preparation, or a compound preparation.

27. The method according to claim 9, wherein the pharmaceutically acceptable salt of the compound represented by Formula I is a hydrochloride salt, hydrobromide salt, hydroiodide salt, nitrate salt, sulfate, hydrogen sulfate, phosphate, hydrogen phosphate, acetate, propionate, butyrate, oxalate, pivalate, adipate, alginate, lactate, citrate, tartrate, succinate, maleate, fumarate, picrate, aspartate, gluconate, benzoate, methanesulfonate, ethanesulfonate, benzenesulfonate, p-toluenesulfonate, or embonate of the compound.

28. The method according to claim 27, wherein the pharmaceutically acceptable salt of the compound represented by Formula I is a methanesulfonate of the compound.


SEQUENCE LISTING (2 sequences)

SEQ ID NO: 1 - DNA, 21 bp, Artificial Sequence
  Primer
caatggtttaacaggcacagg

SEQ ID NO: 2 - DNA, 21 bp, Artificial Sequence
  Primer
ctcaagtgtctgtggatcacg